(19) United States
(12) Patent Application Publication
(10) Pub. No.: US 2011/0201054 A1
(43) Pub. Date: Aug. 18, 2011

(54) Process for improved recovery of fermentation products from intracellular and extracellular presence

(75) Inventors: Dilip S. Mehta, Mumbai (IN); Anselm de Souza, Mumbai (IN)

(73) Assignee: Viridis Biopharma Pvt Ltd

(21) Appl. No.: 12/661,547

(22) Filed: Mar. 18, 2010

(30) Foreign Application Priority Data
Feb. 18, 2010 (IN) 380/MUM/2009

(51) Int. Cl. C12P 7/26; C12N 9/00; C12P 19/42; C12P 7/62; C12P 17/06

(52) U.S. Cl. 435/86; 435/183; 435/148; 435/135; 435/125

(57) ABSTRACT

The present invention increases yield of the target fermented molecules by spray drying the fermentation broth and reducing the process volume to nearly one tenth the original. Thus, none of the target molecules are lost and requirements of the solvent and the other conventional method process equipments and processing materials are reduced considerably. Product recovery is improved. A major saving in the waste water treatment offsets spray drying and offers better process economics.

### RELATED APPLICATION

This application claims priority of the Indian Complete Patent application 380/MUM/2009 filed 18 Feb. 2010, and which is incorporated herein in its entirety.

### FIELD OF THE INVENTION

This invention relates to methods for isolating desired fermentation bioproducts of reactions conducted in aqueous fermentation broths. The invention further provides a process for isolation of Vitamin K2-7 (Mn-7), Vitamin B₁₂ and statins produced by fermentation process including pravastatin, compactin and lovastatin.

### BACKGROUND OF THE INVENTION

The demand for efficient production methods has increased vastly in response to rapid advances in the use of fermentation products in food and pharmaceutical industry. The main aims for industrial biotechnologists is to devise and select processes to obtain a high quality product at an efficient recovery rate using minimum plant investment operated at minimal costs. Unfortunately, recovery costs of microbial products may vary as low as 15% to as high as 70% of the total manufacturing costs. Blanch et al [1] estimate typically 50-70% of the total production cost in classical processes is due to downstream processing, whereas in fermentation that employs recombinant DNA, the fraction can reach up to 80-90%. This large percentage is often due to separation and purification of the fermentation product, as the product formed is a small percentage compared to impurities like microbial biomass, nutrients and metabolites present in the fermentation broth.

The chosen process and therefore its relative costs will depend on the specific product. The present practice gives a total cost of 15% for industrial ethanol, 20-30% for bulk Penicillin G and up to 70% for enzymes. 1,3-Propanediol and 2,3-butanediol are two promising chemicals which have a wide range of applications and can be biologically produced. The separation of these diols from fermentation broth contributes to more than 50% of the total costs in their microbial production [2].

If a fermentation broth is analyzed at the time of harvesting it will be discovered that the
1. The product of interest may reside intracellular in higher concentration and extracellular in low concentrations.
2. The product of interest may reside extracellular in higher concentration and intracellular in low concentrations.
3. The product of interest may reside non-ignorable in both phases.

Fermentation broths are complex aqueous mixtures of cells, soluble extracellular products, intracellular products, and converted substrate or unconvertible components. The particular separation techniques useful for any given bioprocess depend not only on the location of the product (intracellular vs. extracellular) and its size, charge and solubility, but also on the scale of the process itself and the product value. For example, chromatography is generally useful for high-value pharmaceuticals or biologicals, such as hormones, antibodies and enzymes, but is expensive and difficult to scale up.

Fermentation recovery poses difficult challenges because of very dilute solutions of the target molecule and their separation from byproducts with very similar properties. Hence product recovery, from fermentation broth, requires multistep more often than not and low yield steps compound the decreasing effect on the overall yield. Table 1 provides a demonstration of a general downstream process yield. The increasing loss in the recovery process is highlighted in Table 1.

TABLE 1
Recovery at each step in a multiple step recovery process

| | | | | | | | |
|---|---|---|---|---|---|---|---|
| Yield for each step | 95 | 90 | 85 | 80 | 70 | 60 | 50 |
| Overall yield if just 1 step | 95 | 90 | 85 | 80 | 70 | 60 | 50 |
| Overall yield if just 2 step | 90 | 81 | 72 | 64 | 48 | 36 | 25 |
| Overall yield if just 3 step | 85 | 72 | 61 | 51 | 34 | 21 | 12 |
| Overall yield if just 4 step | 81 | 65 | 52 | 40 | 24 | 12 | 6 |
| Overall yield if just 5 step | 77 | 59 | 44 | 32 | 16 | 7 | 3 |
| Overall yield if just 6 step | 73 | 53 | 37 | 26 | 11 | 4 | 1 |
| Overall yield if just 7 step | 69 | 47 | 32 | 20 | 8 | 2 | 0 |
| Overall yield if just 8 step | 66 | 43 | 27 | 16 | 5 | 1 | 0 |
| Overall yield if just 9 step | 63 | 38 | 23 | 13 | 4 | 1 | 0 |
| Overall yield if just 10 step | 59 | 34 | 19 | 10 | 2 | 0 | 0 |

Conventional recovery strategies have included cell lysing, centrifugations, steam stripping, distillation, membrane filtration, pervaporation, ion exchange chromatography, liquid-liquid extraction, and reactive extraction and more. No single method has proved to be simple and efficient, and improvements are especially needed with regard to yield, purity, and energy consumption. Many different compounds can be produced by using microorganisms or enzymes. An important element in the design of a viable biotechnological process is the selection of an economical and efficient separation train.

The industrial production of beta-lactam antibiotics by fermentation over the past 50 years is one of the outstanding examples of biotechnology. Today, the beta-lactam antibiotics, particularly penicillins and cephalosporins, represent the world's major biotechnology products with worldwide dosage form sales of approximately 15 billion US dollars or approximately 65% of the total world market for antibiotics [3]. Such large fermentation productions and the newer ones employing recombinant bacteria, mammalian cells, and transgenic animals for the production of high-value therapeutic proteins call for refinements in recovery strategies. There have been several such improvements.

Human serum albumin (HSA), required in large-scale production due to its use in a number of diseases in high dosage, has been produced through precipitation methods. Lu and Su [4] have come up with a new process involving STREAMLINE expanded bed adsorption to directly capture the target product from the fermentation broth. This novel process eliminates the need to separate the cells by centrifugation or membrane filtration. Garcia has reviewed expanded bed adsorption and polymeric adsorbents [5].

The present invention relates to separation of molecules that are stable at high drying temperatures for a short period. Besides many fermented industrially important molecules suitable for this invention, our target processes are for the purification of statins, Vitamin B12 and Vitamin K2. These are large productions and will benefit from the current invention.

After fermentation, Pravastatin is separated from the fermentation broth by acidifying the broth to a pH of 3 and extracting pravastatin and other non-hydrophilic organics with ethyl acetate, followed by washing with brine. The pravastatin is lactonized, neutralized and dried. The residue is purified by reverse-phase HPLC. U.S. Pat. No. 5,942,423 [6] relates to isolation of analytical scale quantities by HPLC.

U.S. Pat. No. 5,712,130 [7] describes a process for the extraction of lovastatin from the fermentation broth with butyl acetate followed by centrifugation, separation of aqueous phase, concentration, and double crystallization.

U.S. Pat. No. 6,387,258 [8] describes purification of statin compounds by alkaline pretreatment and an alkaline purification. Both, hydrophobic organic extraction and crystallization are used to recover pure statin compound.

U.S. Pat. No. 6,444,452 [9] relates to an improvement in statin recoveries through enrichment by salt formation and re-extraction of pravastatin.

U.S. Pat. No. 5,378,621 [10] reports a method for killing fungal cells without lysing in fermentation process in order to prepare the fermentation mixture for processing to recover or extract an extracellularly expressed enzyme from the fermentation mixture. The method envisions adding a mineral acid to the mixture to a pH of less than 2.79 and then achieving a complete kill by adding acetic acid to the mixture.

Submerged fermentation involves the production of biomolecules in fermenters, volumes ranging from 10 KL to 200 KL. The molecules synthesized therein are in a dilute and impure form and have to be concentrated and purified to acceptable pharmacopeias levels. The process of extraction and purification is accomplished either by solvent extraction of the fermented broth or adsorption of the biomolecule on ion exchange columns or precipitation with salts in the case of proteins. In each of the above processes cost of production increases due to use of large volume of solvents and ion exchange resins, more importantly large volume of waste are generated which have to be treated before discharge. Presented here is a novel method for extraction and purification of biologically fermented products by spray drying the fermented broth to 1/10^{th} the volume followed by solvent extraction and purification.

### SUMMARY

The present invention relates to the process of recovery of biomolecules from a fermentation broth. It also relates to the recovery of vitamin K2-7, vitamin B12, lovastatin and enzymes from a fermentation broth.

In one aspect the invention provides a process of recovery of biomolecules from a fermentation broth using sequential steps of:
a. fermentation using conventional process;
b. spray drying the mixture of step (b) into a drying chamber;
c. subjecting the spray dried material obtained in step (c) to solvent extraction and further purification steps

In another aspect of the invention the aforementioned biomolecule is selected from a group consisting of enzyme, vitamin K2-7, vitamin B12 and statin. The statin is selected from a group consisting of pravastatin, compactin and lovastatin.

In yet another aspect, the invention provides a process recovery of vitamin K2-7 from a fermentation broth comprising the following sequential steps:
a) fermentation using conventional process;
b) adding an inert carrier to the fermentation broth obtained in step (a) and thoroughly mixing it;
c) spray drying the mixture of step (b) using a rotary atomizer into a drying chamber;
d) extraction of vitamin K2-7 from the spray dried powder obtained in step (c) using solvent extraction.

The process of recovery of vitamin K2-7 from a fermentation broth involves use of a drying chamber at a temperature of 60° C.-80° C., a rotary atomizer at an inlet temperature of 120° C.-260° C., an inert carrier consisting of NaCl or dextrin and a solvent of step (d) selected from a group comprising hexane, toluene and methanol.

The invention also provides a process of recovery of vitamin B12 from a fermentation broth comprising the following sequential steps:
a) fermentation using conventional process;
b) conversion of cobalamin present in the fermentation broth of step (a) to vitamin B12;
c) addition of 2-5% dextrin to the fermentation broth obtained in step (b) and thoroughly mixing it;
d) spray drying the mixture of step (c) into a drying chamber;
e) extraction of vitamin B12 from the spray dried powder obtained in step (d) with a solvent or solvent mixture in the recycle mode.

The process of recovery of vitamin B12 from a fermentation broth involves use of a drying chamber at a temperature of 110° C.-160° C., a solvent of step (e) selected from a group comprising chloroform, butanol and carbon tetrachloride and a solvent mixture of step (e) which is 2:1 chloroform:butanol.

The invention further provides a process of recovery of lovastatin from a fermentation broth comprising the following sequential steps:
a) fermentation using conventional process;
b) acidification of the broth of step (a) and separation of the mycelial cake using a filter press;
c) addition of 2% dextrin to the fermentation broth obtained in step (b) and thoroughly mixing it;
d) spray drying the mixture of step (c) into a drying chamber;
e) extraction of lovastatin from the spray dried powder obtained in step (d) with a solvent.

The process of recovery of lovastatin from a fermentation broth involves use of a solvent of step (e) selected from a group comprising toluene, methanol and acetone. The temperature of toluene used is 60° C.-70° C.

The invention also further provides a process of recovery of enzymes from a fermentation broth comprising the following sequential steps:
a) fermentation using conventional process;
b) addition of 5% filler;
c) spray drying the mixture of step (c) into a drying chamber;
d) extraction of enzyme from the spray dried powder obtained in step (c) using ammonium sulphate precipitation.

The process of recovery of enzymes from a fermentation broth involves use of a drying chamber at a temperature of 65° C., spray nozzle with an inlet temperature of 100° C.-140° C. and a filler which is filler is salt or white dextrin.

### DETAILED DESCRIPTION OF THE INVENTION

Present technology for extraction of fermentation based bioproducts involves handling of large volume of broth containing microbial cells, metabolites and unused nutrients. Fermentation technologists normally apply the principle of “Remove the major impurity first” in extraction of the desired bioproducts. In all submerged fermentations the major impurity is water which normally comprises more than 90% of the volume to be processed. The remaining 10% consists of biomass, nutrients, metabolites and the product of interest.

According to the present invention it is demonstrated that removal of water through spray drying of the broth greatly enhances the process economics by:
1. Reduction of material to be processed to 1/10^{th} the original load.
2. Extraction of this reduced load with relatively small quantities of solvent.
3. Further purification of the extracted materials using conventional purification techniques.
4. Treatment of larger quantities of waste-water is a major concern not only in terms of energy but also odor pollution. Odor pollution is a major cause of concern in fermentation effluent treatment plants due to the large volumes and insufficient aeration leading to anaerobic conditions causing offensive odor.

The following specific examples illustrate the process of the present invention but they are not limited to the scope of the invention. For the sake of comparison and appreciation of the novel process both conventional and proposed processes are elucidated.

### Example 1

### Production of Menaquinone-7 by Bacillus Subtilis

### Conventional Process:

### Fermentation:

Bacillus subtilis natto is grown in a medium containing 10.0% soyabean extract, 5.0% glycerol, 0.5% yeast extract and 0.05% K₂HPO₄ Submerged fermentation was carried out with aeration at 37° C. for 24 h followed by static conditions for 120 h [11].

Purification: 2-Propanol (1.2 L) and n-Hexane (2.4 L) is added to 1.0 L of culture broth of Bacillus subtilis. This mixture is vigorously agitated and allowed to settle. The n-hexane layer is removed which contains vitamin K2-7. A second wash of n-hexane is given if necessary to completely extract the vitamin K2-7.

The aqueous layer containing water, cells and 2-propanol is stripped of 2-Propanol and then sent to the effluent treatment plant for biological treatment.

The hexane layer which is normally 2.5 times the broth volume is concentrated and the crude vitamin K2-7 obtained is purified by silica gel chromatography.

### Novel Process:

Fermentation is carried out by the conventional process. At the end of the fermentation cycle to 1.0 L of broth 50.0 gms of an inert carrier like NaCl or white dextrin is added and thoroughly mixed to dissolve. This material is then spray dried using a rotary atomizer at an inlet temperature of 120-260° C. and a chamber temperature of 60-80° C. Approximately 55.0 gms of spray dried powder is obtained from 1.0 L broth.

This spray dried powder contains 100% of the vitamin K2-7 present in the broth. In other words, this first step recovery is 100%. Extraction of the K2-7 can be easily done using solvents like hexane, toluene or methanol. 250 ml solvent is sufficient to extract >90% of vitamin K2-7 from the 55.0 gms of dry powder as the solvent can be recycled through the powder. This is not possible in the conventional process since both phases are aqueous.

The solvent rich material which is normally 25% of the original broth volume is then taken for further purification by conventional process described earlier.

The advantage of this process can be easily appreciated as
1) Only a single solvent is used in extraction;
2) 10% of solvent is used in extraction compared to conventional process;
3) Less energy is required to distill off the solvent;
4) Extraction efficiencies are higher
5) No liquid waste is generated.

In the prior art [12] the inventors propose spray drying of the broth or freeze drying directly and packing the spray dried material into sachets under vacuum. This method is not suitable commercially since:
1) The spray dried material will contain a very high viable count of microbial cells (>10¹³ CFU/g of product. Limits of bacterial count for nutraceuticals are less than 10,000 CFU/g.
2) On opening the sachet for formulation the product will be exposed to air as it is no longer under vacuum. Thus material is susceptible to oxidation and degradation.
3) Product obtained by this method is crude and contains a high level of impurities with offensive odor.

### Example 2

### Production of Cyanocobalamin (Vitamin B₁₂)

### Conventional Process:

### Fermentation

A strain of Pseudomonas denitrificans is grown in a nutrient medium containing Beet Molasses 120 g/L, CaCl₂ 0.5 g/L, 5,6 Dimethybenzimidazole 0.01 g/L, FeSO₄—0.2%, ZnSO₄—0.5%, NaMoO₄—0.001%. The fermentation was allowed to proceed for 120 h under aeration condition of 0.5 VVM air (volume of air per volume of medium per minute) and suitable agitation. At 120 h when maximum productivity is achieved the broth is harvested.

### Purification

For 80 KL of fermentation broth, 150 L of Concentrated H₂SO₄ is slowly added to adjust pH to 2.8. The cobalamin formed in fermentation is converted to cyano cobalamin by the addition of 8 Kg NaCN and 200 Kgs NaN0₂ and heating at 80-100° C. for 10 mins.

This broth is then loaded on 3 ion exchange columns in series containing a cation exchanger like Amberlite IRC-50, in the acidic cycle. The spent broth from the 3^{rd} and last column in series is sent to the effluent treatment. Vitamin B₁₂ from the column is eluted by raising the pH by passing 5 N liquor ammonia. The eluate containing rich vitamin B₁₂ is concentrated and further purified by solvent extraction using chloroform:butanol 2:1 The solvent is evaporated and vitamin B₁₂ is back extracted into water phase, and crystallized from the solution using acetone [13].

### Novel Process:

After conversion of the cobalamin to cyanocobalamin the broth is spray dried using 2-5% dextrin as a carrier. The spray dried inlet temperature is maintained at 110-160° C. and chamber temperature at 65° C. Approximately 1.6 to 4.0 MT of Vitamin B₁₂ spray dried powder will be obtained. This Vitamin B₁₂ from the powder can be extracted using a solvent mixture of 2:1 chloroform:butanol in the recycle mode. Approximately 1 KL of solvent mixture is required for 1 MT of powder. The solvent is recovered and Vitamin B₁₂ is back extracted into the aqueous phase and further purified conventionally.

The advantages of the processes are:
1) 80 KL of broth is reduced to a manageable level of 5 MT;
2) 80 KL of broth has to be loaded on 3 ion exchange columns containing 5 MT of resin each which is eliminated by this procedure;
3) Ammonia and HCl used to elute and regenerate the ion exchange columns are saved. Water for washing columns is also saved;
4) No effluent is generated and hence no treatment is required.

### Example 3

### Lovastatin Production

### Conventional Process:

### Fermentation

A commercial strain of Aspergillus terreus is grown [14] in a medium containing skimmed milk powder 55 g/L, soyabean meal 59 g/L, Yeast extract 2.5 g/L Dextrose 5.0 g/L, Sodium acetate 8.75 g/L, Citric Acid 10 g/L, glycerol 5 g/L, CaCO₃ 6 g/L and Antifoam. On fermentation conditions of temp 28° C., 0.5 VVM aeration and agitation maximum productivity is reached after 11 days.

### Purification

Extraction and Purification of Lovastatin from Fermentation Broth is as Per Flow Chart
Adj. pH of fermented broth to 4.0 with H₂SO₄ (1)
   ↓
Add Toluene 1:1 (2)
   ↓
Stir for 5-6 hrs @ 55-60° C. to convert Mevolonic Acid to Lovastatin (3)
   ↓
Filter through filter press (4)
   ↓
Solvent fractions collected and wash with 10% Sodium Bicarbonate solution (5)
   ↓
Repeat Bicarbonate washing
   ↓
Water wash and Brine wash
   ↓
Chemical Treatment
   ↓
Concentrate by vacuum Distillation
   ↓
Crystallize at Temp 5° C.

### Novel Process:

100 L of broth fermented by conventional process is acidified. The broth passed through a filter press and the mycelial cake separated. Clear filtrate is spray dried using 2% dextrin. Approximately 3 kgs of spray dried powder is obtained which can be extracted with 10 L of hot toluene (60-70° C.). Extraction and Lactonization are monitored by HPLC. Subsequent steps are same as from step 5 in conventional process. The mycelial cake is washed separately. As 20% of total volume is the mycelial cake saving in toluene consumption is more than 50% over conventional process. Further lower washes of bicarbonate are required as toluene volume is lower. Finally there is no effluent generated in the process.

### Example 4

### Production of Enzyme

Industrial enzymes like alkaline proteases are produced by fermentation using a Carbon and Nitrogen source like Gram flour. A typical fermentation medium composition comprises of:
Gram Flour—8 g/L
Sucrose—4 g/L
Yeast Extract—0.5 g/L
Peptone—0.5 g/L
pH—7.0

On maximum enzyme production the enzyme is precipitated from the medium by addition of 60% ammonium sulphate. i.e. for 100 L of broth 6.0 kgs ammonium sulphate is added.

The cake is filtered and concentrated enzyme product is obtained which can be taken for formulation e.g detergent manufacture. The filtrate containing high TDS because of Ammonium Sulphate has to be taken for salt recovery before treatment.

### Novel Process:

In the Novel Process the fermentation broth is first spray dried at a Nozzle temp of 120±20° C. and a chamber temp of 65° C. after adding 5% filler. From 100 L of broth 7-7.5 kg of solid spray dried powder will be obtained. This can then be reconstituted to 20 lts with water and ammonium sulphate added (1.2 kgs) to precipitate the enzyme.

Consequently the savings in this novel process are as follows:
1. Less amount of ammonium sulphate required for precipitation (1.2 kgs vs 6.0 kgs for 100 L broth);
2. Less volume of effluent to recover ammonium sulphate;
3. Lower effluent treatment costs.

We claim:

1) A process of recovery of biomolecules from a fermentation broth comprising the sequential steps of:
a. fermentation using conventional process;
b. spray drying the mixture of step (b) into a drying chamber;
c. subjecting the spray dried material obtained in step (c) to solvent extraction and further purification steps

2) The process of claim 1, wherein step (b) yields a volume reduction to about 1/10^{th} of the original broth volume.

3) The process of extraction as claimed in claim 1, wherein the said biomolecule is selected from a group consisting of enzyme, vitamin K2-7, vitamin B12 and statin.

4) The process of claim 3, wherein the statin is selected from a group consisting of pravastatin, compactin and lovastatin.

5) A process of recovery of vitamin K2-7 from a fermentation broth comprising the following sequential steps:
a) fermentation using conventional process;
b) adding an inert carrier to the fermentation broth obtained in step (a) and thoroughly mixing it;
c) spray drying the mixture of step (b) using a rotary atomizer into a drying chamber;
d) extraction of vitamin K2-7 from the spray dried powder obtained in step (c) using solvent extraction.

6) The process of claim 5, wherein the inert carrier of step (b) consists of NaCl or dextrin.

7) The process of claim 5, wherein the inlet temperature of the rotary atomizer is 120° C.-260° C.

8) The process of claim 5, wherein the temperature of the drying chamber is 60° C.-80° C.

9) The process of claim 5, wherein the solvent used step (d) is selected from a group comprising hexane, toluene and methanol.

10) A process of recovery of vitamin B12 from a fermentation broth comprising the following sequential steps:
a) fermentation using conventional process;
b) conversion of cobalamin present in the fermentation broth of step (a) to vitamin B12;
c) addition of 2-5% dextrin to the fermentation broth obtained in step (b) and thoroughly mixing it;
d) spray drying the mixture of step (c) into a drying chamber;
e) extraction of vitamin B12 from the spray dried powder obtained in step (d) with a solvent or solvent mixture in the recycle mode.

11) The process of claim 10, wherein the temperature of the drying chamber is 110° C.-160° C.

12) The process of claim 10, wherein solvent of step (e) is selected from a group comprising chloroform, butanol and carbon tetrachloride.

13) The process of claim 10, wherein solvent mixture of step (e) is 2:1 chloroform:butanol.

14) A process of recovery of lovastatin from a fermentation broth comprising the following sequential steps:
a) fermentation using conventional process;
b) acidification of the broth of step (a) and separation of the mycelial cake using a filter press;
c) addition of 2% dextrin to the fermentation broth obtained in step (b) and thoroughly mixing it;
d) spray drying the mixture of step (c) into a drying chamber;
e) extraction of lovastatin from the spray dried powder obtained in step (d) with a solvent.

15) The process of claim 14, wherein solvent of step (e) is selected from a group comprising toluene, methanol and acetone.

16) The process of claim 14, wherein the temperature of toluene used is 60° C.-70° C.

17) A process of recovery of enzymes from a fermentation broth comprising the following sequential steps:
a) fermentation using conventional process;
b) addition of 5% filler;
c) spray drying the mixture of step (c) into a drying chamber;
d) extraction of enzyme from the spray dried powder obtained in step (c) using ammonium sulphate precipitation.

18) The process of claim 17, wherein the filler is salt or white dextrin.

19) The process of claim 17, wherein the inlet temperature is 100° C.-140° C.

20) The process of claim 17, wherein the temperature of drying chamber is 65° C.
